(19) United States
(12) Patent Application Publication
(10) Pub. No.: US 2022/0162528 A1
(43) Pub. Date: May 26, 2022

(54) Liquid Dishwashing Detergent Compositions

(71) Applicant: Novozymes A/S, Bagsvaerd (DK)

(72) Inventors: Feng Li, Beijing (CN); Qiang Zhao, Beijing (CN)

(73) Assignee: Novozymes A/S, Bagsvaerd (DE)

(21) Appl. No.: 17/601,019

(22) PCT Filed: Mar. 31, 2020
(86) PCT No.: PCT/CN2020/082399

§ 371 Date: Oct. 1, 2021

(30) Foreign Application Priority Data
Apr. 2, 2019 (CN) PCT/CN2019/080952

(51) Int. Cl. C11D 3/386; C11D 11/00

(52) U.S. Cl. CPC C11D 3/38618; C11D 11/0023

(57) ABSTRACT

The invention provides a liquid detergent composition suitable for hand dishwashing, wherein the composition comprises a protease and provides hand skin care benefits.

### REFERENCE TO A SEQUENCE LISTING

This application contains a sequence listing in computer readable form, which is incorporated herein by reference.

### FIELD OF THE INVENTION

The present invention relates to a liquid detergent composition suitable for hand dishwashing, wherein the composition comprises a protease and provides hand skin care benefits.

### BACKGROUND OF THE INVENTION

Hand dishwashing using conventional liquid detergent compositions is often damaging to the skin and/or results in skin irritation and dryness. Although there have been numerous attempts to develop hand dishwashing detergents that are mild on the skin, there is still a need for detergent compositions that have improved skin mildness while maintaining optimal cleaning performance.

Proteases designed to improve removal of proteinaceous stains are standard ingredients in detergent compositions for laundry and automatic dishwashers, and are found in a variety of different types of compositions such as powders, liquids, pouches, tabs, bars, etc. for machine use. On the other hand, proteases have not found widespread use in hand dishwashing liquids.

Liquid detergent compositions for hand dishwashing comprising a protease have been described in the patent literature, e.g. in WO 95/07971, WO 97/25397, WO 2010/088161, WO 2010/088163, WO 2010/088164 and WO 2012/015852. In many cases, such compositions include ingredients such as emollients, polymers, humectants, and other components designed to provide a pleasing skin feel or otherwise reduce skin irritation and dryness that can accompany hand dishwashing.

The present invention provides liquid detergent compositions containing a protease that have been found to provide substantial skin care benefits, even without the presence of special skin care or moisturizing components.

### SUMMARY OF THE INVENTION

The present invention generally relates to liquid detergent compositions suitable for hand-dishwashing and comprising at least one protease.

In one aspect, the invention relates to liquid detergent compositions comprising at least one protease, wherein use of the compositions, e.g. in hand dishwashing, provides at least one hand skin care benefit selected from corneocyte removal, reduced transepidermal water loss, an improved hand skin barrier, and improved hand skin water retention.

In another aspect, the invention relates to use of a protease for preparing a liquid detergent composition suitable for hand dishwashing, wherein the composition provides at least one hand skin care benefit selected from corneocyte removal, reduced transepidermal water loss, an improved hand skin barrier, and improved hand skin water retention.

In a further aspect, the invention relates to a method for preparing a hand dishwashing detergent composition with skin care benefits, the method comprising adding to the composition at least one protease.

In a further aspect, the invention relates to use of a composition comprising a protease as disclosed herein in a cleaning process, e.g. for hand dishwashing.

In a further aspect, the invention relates to a method of cleaning, e.g. for hand dishwashing or hard surface cleaning, comprising contacting dishes or a hard surface to be cleaned with a detergent composition comprising a protease as described herein under conditions suitable for cleaning the dishes or surface.

### Overview of sequences

SEQ ID NO: 1 is the sequence of the Savinase® protease polypeptide from Bacillus lentus.

SEQ ID NO: 2 is the sequence of the BPN′ protease polypeptide from Bacillus amyloliquefaciens.

SEQ ID NO: 3 is the sequence of the TY145 protease from Bacillus sp.

SEQ ID NO: 4 is the sequence of the protease disclosed in GENESEQP under accession number BER84782.

SEQ ID NO: 5 is the sequence of the Alcalase® protease (also known as subtilisin Carlsberg) from Bacillus licheniformis.

### BRIEF DESCRIPTION OF THE FIGURES

FIG. 1 is an alignment of the amino acid sequences of subtilisin 309 (SEQ ID NO: 1) and subtilisin BPN′ (SEQ ID NO: 2).

### Definitions

Subtilase/protease: The terms “subtilase” and “protease” may be used interchangeably herein and refer to an enzyme that hydrolyses peptide bonds in proteins. This includes any enzyme belonging to the EC 3.4 enzyme group (including each of the thirteen subclasses thereof), and in particular endopeptidases (EC 3.4.21). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

Protease activity: The term “protease activity” means a proteolytic activity (EC 3.4), in particular endopeptidase activity (EC 3.4.21). There are several protease activity types, the three main activity types being: trypsin-like, where there is cleavage of amide substrates following Arg or Lys at P1, chymotrypsin-like, where cleavage occurs following one of the hydrophobic amino acids at P1, and elastase-like with cleavage following an Ala at P1. Protease activity may be determined according to the procedure described in WO 2016/087619.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Variant: The term “variant” means a polypeptide having protease activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.

Fragment: The term “fragment” means a polypeptide having one or more amino acids absent from the amino and/or carboxyl terminus of a mature polypeptide; wherein the fragment has subtilase activity. Such a fragment preferably contains at least 85%, at least 90% or at least 95% of the number of amino acids in SEQ ID NO: 1.

### Conventions for Designation of Variants

For purposes of the present invention, the polypeptide of SEQ ID NO: 2 is used to determine the corresponding amino acid residue number in a variant of SEQ ID NO: 1. The amino acid sequence of a variant of SEQ ID NO: 1 is aligned with SEQ ID NO: 2, and based on the alignment, the amino acid position number corresponding to any amino acid residue in the polypeptide of SEQ ID NO: 1 is determined. See the paragraph “Numbering of amino acid positions/residues” below for further information.

For SEQ ID NO: 3, amino acid position numbers are according to SEQ ID NO: 3.

Identification of the corresponding amino acid residue in another subtilase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

In describing the variants of the present invention, the nomenclature described below is adapted for ease of reference. The accepted IUPAC single letter or three letter amino acid abbreviation is employed. The terms “alteration” or “mutation” may be used interchangeably herein to refer to substitutions, insertions and deletions.

Substitutions. For an amino acid substitution, the following nomenclature is used: Original amino acid, position, substituted amino acid. For example, the substitution of a threonine at position 220 with alanine is designated as “Thr220Ala” or “T220A”. Multiple substitutions may be separated by addition marks (“+”), e.g., “Thr220Ala+Gly229Val” or “T220A+G229V”, representing substitutions at positions 220 and 229 of threonine (T) with alanine (A) and glycine (G) with valine (V), respectively. Multiple substitutions may alternatively be listed with individual mutations separated by a space or a comma. Alternative substitutions in a particular position may be indicated with a slash (“/”). For example, substitution of threonine in position 220 with either alanine, valine or leucine many be designated “T220A/V/L”.

Deletions. For an amino acid deletion, the following nomenclature is used: Original amino acid, position, *. Accordingly, the deletion of threonine at position 220 is designated as “Thr220*” or “T220*”. Multiple deletions may be separated by addition marks (“+”), e.g., “Thr220*+Gly229*” or “T220*+G229*”, or alternatively may be separated by a space or comma. The use of an “X” preceding a position number is as described above for substitutions, e.g. “X131*” means that the amino acid residue at position 131 is deleted.

Insertions. For an amino acid insertion, the following nomenclature is used: Original amino acid, position, original amino acid, inserted amino acid. Accordingly, the insertion of lysine after threonine at position 220 is designated “Thr220ThrLys” or “T220TK”. An insertion of multiple amino acids is designated [Original amino acid, position, original amino acid, inserted amino acid #1, inserted amino acid #2; etc.]. For example, the insertion of lysine and alanine after threonine at position 220 is indicated as “Thr220ThrLysAla” or “T220TKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 220 | 220 220a 220b |
| T | T-K-A |

Multiple alterations. Variants comprising multiple alterations are separated by addition marks (“+”), e.g., “Arg170Tyr+Glyl95Glu” or “R170Y+G195E” representing a substitution of arginine and glycine at positions 170 and 195 with tyrosine and glutamic acid, respectively. Multiple alterations may alternatively be listed with individual mutations separated by a space or a comma.

A combination of e.g. a substitution and an insertion may be denoted as follows: S99AD, which represents substitution of a serine residue in position 99 with an alanine residue as well as insertion of an aspartic acid residue.

Different alterations. Where different alterations can be introduced at a position, the different alterations may be separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

Different alterations in a position may also be indicated with a slash (“/”), for example “T220A/V/L” as explained above. Alternatively, different alterations may be indicated using brackets, e.g., Arg170[Tyr, Gly] or in one-letter code R170 [Y,G].

Numbering of amino acid positions/residues. Amino acid position numbers as used herein are based on the numbering of the BPN' polypeptide of SEQ ID NO: 2. Thus, amino acid positions of a parent protease polypeptide having e.g. SEQ ID NO: 1 are those of the corresponding positions of SEQ ID NO: 2. This numbering system is conventional in the art, where position numbers used for subtilisin proteases in the patent literature are often based on the corresponding position numbers of BPN′.

Specifically, the numbering is based on the alignment in Table 1 of WO 89/06279, which shows an alignment of five proteases, including the mature polypeptide of the subtilase BPN′ (BASBPN) sequence (sequence c in the table) and the mature polypeptide of subtilisin 309 from Bacillus lentus, also known as Savinase® (BLSAVI) (sequence a in the table).

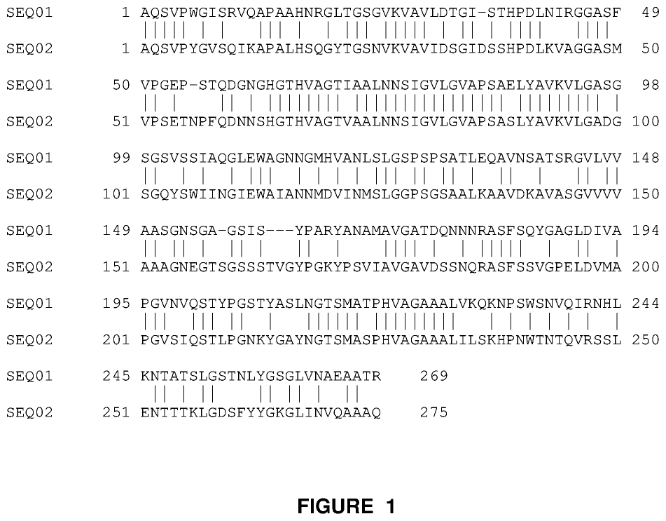

The accompanying FIG. 1 is provided for reference purposes and shows an alignment between SEQ ID NO: 1 and SEQ ID NO: 2, based on Table 1 of WO 89/06279, from which position numbers corresponding to positions of SEQ ID NO: 2 may be readily determined. For a parent protease other than SEQ ID NO: 1, the amino acid sequence of another protease may be similarly aligned with SEQ ID NO: 2 to determine amino acid position numbers corresponding to the numbering of SEQ ID NO: 2.

For SEQ ID NO: 3, position numbers are based on SEQ ID NO: 3.

### DETAILED DESCRIPTION OF THE INVENTION

As explained above, the present invention relates to liquid detergent compositions suitable for hand dishwashing, wherein the compositions comprise at least one protease and can provide one or more skin care benefits.

In a particular aspect, the invention relates to a liquid detergent composition suitable for hand dishwashing, comprising at least one protease, wherein the protease is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2.

In one embodiment of this aspect, the protease is a variant of the polypeptide of SEQ ID NO: 1 comprising 3, 4, 5, 6 or all of the substitutions selected from the group consisting of S9E, N43R, N76D, Q206L, Y209W, S259D and L262E, wherein position numbers are based on the numbering of SEQ ID NO: 2, for example a variant having at least 80%, at least 85%, at least 90% or at least 95% sequence identity to SEQ ID NO: 1. In one embodiment, the protease comprises or consists of the polypeptide of SEQ ID NO: 1 with 3, 4, 5, 6 or all of said substitutions.

In another embodiment, the protease is a variant of the polypeptide of SEQ ID NO: 1 comprising 3, 4, 5, 6, 7, 8 or all of the substitutions selected from the group consisting of S9E, N43R, N76D, V205I, Q206L, Y209W, S259D, N261W and L262E, wherein position numbers are based on the numbering of SEQ ID NO: 2, for example a variant having at least 80%, at least 85%, at least 90% or at least 95% sequence identity to SEQ ID NO: 1. In one embodiment, the protease comprises or consists of the polypeptide of SEQ ID NO: 1 with 3, 4, 5, 6, 7, 8 or all of said substitutions.

In another embodiment, the protease variant comprises or consists of SEQ ID NO: 1 with the substitutions S9E, N43R, N76D, V205I, Q206L, Y209W, S259D, N261W and L262E, wherein position numbers are based on the numbering of SEQ ID NO: 2, for example a variant having at least 80%, at least 85%, at least 90% or at least 95% sequence identity to SEQ ID NO: 1. In a preferred embodiment, the protease comprises or consists of the polypeptide of SEQ ID NO: 1 with the substitutions S9E, N43R, N76D, V205I, Q206L, Y209W, S259D, N261W and L262E.

In another embodiment, the protease is a variant of the polypeptide of SEQ ID NO: 1 comprising 3, 4, 5, 6, 7, 8, 9, 10 or all of the substitutions selected from the group consisting of S9E, N43R, N76D, N185E, S188E, Q191N, A194P, Q206L, Y209W, S259D and L262E, wherein position numbers are based on the numbering of SEQ ID NO: 2, for example a variant having at least 80%, at least 85%, at least 90% or at least 95% sequence identity to SEQ ID NO: 1. In one embodiment, the protease comprises or consists of the polypeptide of SEQ ID NO: 1 with 3, 4, 5, 6, 7, 8, 9, 10 or all of said substitutions.

In another embodiment, the protease comprises or consists of SEQ ID NO: 1 with the substitutions S9E, N43R, N76D, N185E, S188E, Q191N, A194P, Q206L, Y209W, S259D and L262E, wherein position numbers are based on the numbering of SEQ ID NO: 2, for example a variant having at least 80%, at least 85%, at least 90% or at least 95% sequence identity to SEQ ID NO: 1. In one embodiment, the protease comprises or consists of the polypeptide of SEQ ID NO: 1 with the substitutions S9E, N43R, N76D, N185E, S188E, Q191N, A194P, Q206L, Y209W, S259D and L262E.

In another aspect, the invention relates to a liquid detergent composition suitable for hand dishwashing, comprising at least one protease, wherein the protease is selected from the group consisting of:

(a) the polypeptide of SEQ ID NO: 1;

(b) a variant of the polypeptide of SEQ ID NO: 1 with the mutation S99SE (i.e. insertion of E after position 99), wherein position numbers are based on the numbering of SEQ ID NO: 2;

(c) a variant of the polypeptide of SEQ ID NO: 1 with the mutation S99AD (i.e. substitution of S99 with A, followed by insertion of D), wherein position numbers are based on the numbering of SEQ ID NO: 2;

(d) a variant of the polypeptide of SEQ ID NO: 1 with the substitutions Y167A+R170S+ A194P, wherein position numbers are based on the numbering of SEQ ID NO: 2;

(e) a variant of the polypeptide of SEQ ID NO: 1 with the substitutions S99D+S101R/E+ S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;

(f) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and

(g) the polypeptide of SEQ ID NO: 4.

In one embodiment, use of the composition comprising a protease as described above in a hand dishwashing provides at least one hand skin care benefit selected from corneocyte removal, reduced transepidermal water loss, an improved hand skin barrier, and improved hand skin water retention.

Another aspect of the invention relates to use of a protease for preparing a liquid detergent composition suitable for hand dishwashing, wherein the protease provides at least one hand skin care benefit selected from corneocyte removal, reduced transepidermal water loss, an improved hand skin barrier, and improved hand skin water retention. In a preferred embodiment, the protease of this aspect is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2. The protease of this aspect may thus, for example, comprise or consist of the polypeptide of SEQ ID NO: 1 with the substitutions S9E, N43R, N76D, V2051, Q206L, Y209W, S259D, N261W and L262E; or it may comprise or consist of the polypeptide of SEQ ID NO: 1 with the substitutions S9E, N43R, N76D, N185E, S188E, Q191N, A194P, Q206L, Y209W, S259D and L262E.

A preferred protease for use in this aspect of the invention is one which comprises or consists of the polypeptide of SEQ ID NO: 1 with the substitutions S9E, N43R, N76D, V205I, Q206L, Y209W, S259D, N261W and L262E.

In another embodiment of this aspect of the invention, the protease may be selected from the group consisting of:

(a) the polypeptide of SEQ ID NO: 1;

(b) a variant of the polypeptide of SEQ ID NO: 1 with the mutation S99SE, wherein position numbers are based on the numbering of SEQ ID NO: 2;

(c) a variant of the polypeptide of SEQ ID NO: 1 with the mutation S99AD, wherein position numbers are based on the numbering of SEQ ID NO: 2;

(d) a variant of the polypeptide of SEQ ID NO: 1 with the substitutions Y167A+R170S+ A194P, wherein position numbers are based on the numbering of SEQ ID NO: 2;

(e) a variant of the polypeptide of SEQ ID NO: 1 with the substitutions S99D+S101R/E+ S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;

(f) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and

(g) the polypeptide of SEQ ID NO: 4.

As noted above, the liquid detergent compositions of the invention containing a protease have been found to provide substantial skin care benefits, even without the presence of special skin care or moisturizing components. Thus, in one embodiment, the compositions of the invention may optionally be without e.g. emollients, moisturizers, polymers, humectants, and other components that are otherwise used in hand dishwashing liquids to provide skin care benefits or to e.g. reduce skin irritation and dryness that can accompany hand dishwashing.

Another aspect of the invention relates to a method for preparing a hand dishwashing detergent composition with skin care benefits, or for providing skin care benefits to a hand dishwashing detergent composition, the method comprising adding to the composition at least one protease. The skin care benefits preferably comprise at least one hand skin care benefit selected from corneocyte removal, reduced transepidermal water loss, an improved hand skin barrier, and improved hand skin water retention. In a preferred embodiment, the protease of this aspect is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2. The protease of this aspect may thus, for example, comprise or consist of the polypeptide of SEQ ID NO: 1 with the substitutions S9E, N43R, N76D, V205I, Q206L, Y209W, S259D, N261W and L262E; or it may comprise or consist of the polypeptide of SEQ ID NO: 1 with the substitutions S9E, N43R, N76D, N185E, S188E, Q191N, A194P, Q206L, Y209W, S259D and L262E.

A preferred protease for use in this aspect of the invention is one which comprises or consists of the polypeptide of SEQ ID NO: 1 with the substitutions S9E, N43R, N76D, V205I, Q206L, Y209W, S259D, N261W and L262E.

In another embodiment of this aspect of the invention, the protease may be selected from the group consisting of:

(a) the polypeptide of SEQ ID NO: 1;

(b) a variant of the polypeptide of SEQ ID NO: 1 with the mutation S99SE, wherein position numbers are based on the numbering of SEQ ID NO: 2;

(c) a variant of the polypeptide of SEQ ID NO: 1 with the mutation S99AD, wherein position numbers are based on the numbering of SEQ ID NO: 2;

(d) a variant of the polypeptide of SEQ ID NO: 1 with the substitutions Y167A+R170S+ A194P, wherein position numbers are based on the numbering of SEQ ID NO: 2;

(e) a variant of the polypeptide of SEQ ID NO: 1 with the substitutions S99D+S101R/E+ S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;

(f) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and

(g) the polypeptide of SEQ ID NO: 4.

Another aspect of the invention relates to the use of a liquid detergent composition described herein comprising at least one protease in a cleaning process. In this aspect the cleaning process is typically hand dishwashing, although other uses are also possible, in particular other manual cleaning processes, including hard surface cleaning and hand laundry. In a preferred embodiment, the protease of this aspect is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2. The protease of this aspect may thus, for example, comprise or consist of the polypeptide of SEQ ID NO: 1 with the substitutions S9E, N43R, N76D, V205I, Q206L, Y209W, S259D, N261W and L262E; or it may comprise or consist of the polypeptide of SEQ ID NO: 1 with the substitutions S9E, N43R, N76D, N185E, S188E, Q191N, A194P, Q206L, Y209W, S259D and L262E.

A preferred protease for use in this aspect of the invention is one which comprises or consists of the polypeptide of SEQ ID NO: 1 with the substitutions S9E, N43R, N76D, V205I, Q206L, Y209W, S259D, N261W and L262E.

In another embodiment of this aspect of the invention, the protease may be selected from the group consisting of:

(a) the polypeptide of SEQ ID NO: 1;

(b) a variant of the polypeptide of SEQ ID NO: 1 with the mutation S99SE, wherein position numbers are based on the numbering of SEQ ID NO: 2;

(c) a variant of the polypeptide of SEQ ID NO: 1 with the mutation S99AD, wherein position numbers are based on the numbering of SEQ ID NO: 2;

(d) a variant of the polypeptide of SEQ ID NO: 1 with the substitutions Y167A+R170S+ A194P, wherein position numbers are based on the numbering of SEQ ID NO: 2;

(e) a variant of the polypeptide of SEQ ID NO: 1 with the substitutions S99D+S101R/E+ S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;

(f) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and

(g) the polypeptide of SEQ ID NO: 4.

Another aspect of the invention relates to a method of cleaning, e.g. for hand dishwashing or hard surface cleaning, comprising contacting dishes or a hard surface to be cleaned with a detergent composition comprising at least one protease as described herein under conditions suitable for cleaning the dishes or surface. In a preferred embodiment, the protease of this aspect is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2. The protease of this aspect may thus, for example, comprise or consist of the polypeptide of SEQ ID NO: 1 with the substitutions S9E, N43R, N76D, V205I, Q206L, Y209W, S259D, N261W and L262E; or it may comprise or consist of the polypeptide of SEQ ID NO: 1 with the substitutions S9E, N43R, N76D, N185E, S188E, Q191N, A194P, Q206L, Y209W, S259D and L262E.

A preferred protease for use in this aspect of the invention is one which comprises or consists of the polypeptide of SEQ ID NO: 1 with the substitutions S9E, N43R, N76D, V205I, Q206L, Y209W, S259D, N261W and L262E.

In another embodiment of this aspect of the invention, the protease may be selected from the group consisting of:

(a) the polypeptide of SEQ ID NO: 1;

(b) a variant of the polypeptide of SEQ ID NO: 1 with the mutation S99SE, wherein position numbers are based on the numbering of SEQ ID NO: 2;

(c) a variant of the polypeptide of SEQ ID NO: 1 with the mutation S99AD, wherein position numbers are based on the numbering of SEQ ID NO: 2;

(d) a variant of the polypeptide of SEQ ID NO: 1 with the substitutions Y167A+R170S+ A194P, wherein position numbers are based on the numbering of SEQ ID NO: 2;

(e) a variant of the polypeptide of SEQ ID NO: 1 with the substitutions S99D+S101R/E+ S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;

(f) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and

(g) the polypeptide of SEQ ID NO: 4.

It will be apparent that in any of the aspects of the invention described herein, including but not limited to use of a protease for preparing a liquid detergent composition suitable for hand dishwashing, a method for preparing a hand dishwashing detergent composition with skin care benefits or for providing skin care benefits to a hand dishwashing detergent composition by adding to the composition at least one protease, use of a liquid detergent composition comprising at least one protease in a cleaning process, and a method of cleaning using the liquid detergent composition, the protease may be any of the proteases or variants that are described in detail in the present description and claims.

In one embodiment of any of the aspects disclosed herein, the hand skin care benefit comprises enhanced corneocyte removal.

In one embodiment of any of the aspects disclosed herein, the hand skin care benefit comprises reduced transepidermal water loss and/or an improved hand skin barrier.

In one embodiment of any of the aspects disclosed herein, the hand skin care benefit comprises improved hand skin water retention.

A liquid detergent composition of the invention suitable for hand dishwashing will typically include the protease in an amount of from about 0.001% w/w to about 4% w/w, such as about 0.01% w/w to about 2% w/w, such as about 0.05% w/w to about 1% w/w, such as about 0.1% w/w to about 0.5% w/w, wherein percentages are enzyme protein by weight of the composition.

In a preferred embodiment, the liquid detergent compositions of the invention not only provide hand skin care benefits as described herein, but also provide good cleaning performance, meaning cleaning performance that it at least as good as or, preferably, better than a corresponding liquid detergent composition without the protease. Since proteases are well-known for their protein stain removal properties in e.g. laundry and automatic dishwashing detergents, it is expected that the compositions of the invention will normally provide cleaning benefits as well as hand skin care benefits.

In another embodiment, the protease is highly stable, providing the composition with good shelf stability. Non-limiting examples of proteases which have been found to have good stability and which are therefore suitable for use in liquid detergent compositions of the invention that are intended to have good shelf life include: a variant of SEQ ID NO: 1 with the substitutions S9E, N43R, N76D, V205I, Q206L, Y209W, S259D, N261W and L262E; and a variant of SEQ ID NO: 1 with the substitutions S9E, N43R, N76D, N185E, S188E, Q191N, A194P, Q206L, Y209W, S259D and L262E.

In addition to the amino acid alterations specifically disclosed herein, a protease variant in a composition of the invention may comprise additional alterations at one or more other positions. These additional alterations may be of a minor nature, that is typically conservative amino acid substitutions or insertions that do not significantly affect the folding and/or activity of the protein, and which do not alter the net formal charge as described herein; small deletions, typically of 1-10 or 1-5 amino acids; or small amino- or carboxyl-terminal extensions. Another possible alteration is a truncation of the N-terminal and/or C-terminal, for example where one or both are truncated by 1-5 amino acids, resulting in a fragment of the protease variant that maintains protease activity.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, in The Proteins, Academic Press, New York. Common conservative substitution groups include, but are not limited to: G=A=S; I=V=L=M; D=E; Y=F; and N=Q (where e.g. “G=A=S” means that these three amino acids may be substituted for each other).

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Detergent Compositions

For purposes of the present invention the detergent composition is a liquid, in particular one that is suitable for hand dishwashing, although it is also contemplated that compositions of the invention may also be used for other purposes such as hard surface cleaning or hand laundry.

Liquid Composition

The liquid detergent composition of the invention is typically formulated as a liquid dishwashing detergent composition suitable for hand dishwashing as is well-known in the art.

In addition to the protease, the compositions will typically contain from 30% to 90% by weight of an aqueous liquid carrier in which the other components of the composition are dissolved, dispersed or suspended. Preferably, the aqueous liquid carrier will comprise from 45% to 70%, more preferably from 45% to 65% by weight of the composition. In one preferred embodiment, the aqueous liquid carrier is water. Alternatively, the aqueous liquid carrier may contain other materials which are liquid, or which dissolve in the liquid carrier, at room temperature (about 20-25° C.) and which may also serve other functions besides that of an inert filler. Such materials can include, for example, hydrotropes and solvents as discussed in more detail below.

The liquid dishwashing composition may have any suitable pH value. Preferably, the pH of the composition is adjusted to between about 6 and 10, preferably between about 7 and 9, such as about 7 or about 8. The pH of the composition can be adjusted using pH modifying ingredients known in the art.

Surfactants

The compositions of the invention preferably comprise at least one surfactant selected from nonionic, anionic, cationic surfactants, amphoteric, zwitterionic, semi-polar nonionic surfactants, and mixtures thereof. Surfactants may be included in an amount of from about 1% to about 50% by weight, preferably from about 5% to about 40% by weight, more preferably from about 5% to about 30% by weight, such as from about 10% to about 20% by weight of the liquid detergent composition. Non-limiting examples of suitable surfactants are discussed below.

In one embodiment, an efficient but mild surfactant system may comprise from about 4% to about 40%, preferably about 6% to about 32%, more preferably about 11% to about 25%, and most preferably about 11% to about 18% by weight of the total composition of an anionic surfactant and optionally no more than about 15%, preferably no more than about 10%, more preferably no more than about 5% by weight of the total composition, of a sulfonate surfactant.

Suitable anionic surfactants to be used in the compositions and methods of the present invention include sulfate, sulfosuccinates, sulfonate, and/or alkyl ethoxy sulfates; more preferably a combination of alkyl sulfates and/or alkyl ethoxy sulfates with a combined ethoxylation degree less than about 5, preferably less than about 3, more preferably less than about 2.

In an alternative embodiment, the surfactant system may be based on high levels of nonionic surfactant (such as about 10% to about 45%, preferably about 15% to about 40%, more preferably about 20% to about 35% by weight of the total composition), preferably combined with an amphoteric surfactant, and more preferably with a low level of anionic surfactant (such as less than 20%, preferably less than 10%, more preferably less than about 5% by weight of the total composition).

Sulfate Surfactants

Suitable sulfate surfactants for use in the compositions herein include water-soluble salts or acids of C₁₀-C₁₄ alkyl or hydroxyalkyl sulfate and/or ether sulfate. Suitable counterions include hydrogen, alkali metal cations or ammonium or substituted ammonium, preferably sodium.

Where the hydrocarbyl chain is branched, it preferably comprises C₁₄ alkyl branching units. The average percentage branching of the sulfate surfactant is preferably greater than 30%, more preferably from 35% to 80% and most preferably from 40% to 60% of the total hydrocarbyl chains.

The sulfate surfactants may be selected from C₈-C₂₀ primary, branched-chain and random alkyl sulfates (AS); C₁₀-C₁₈ secondary (2,3) alkyl sulfates; C₁₀-C₁₈ alkyl alkoxy sulfates (AExS) wherein x is preferably from 1 to 30; C₁₀-C₁₈ alkyl alkoxy carboxylates, preferably comprising 1-5 ethoxy units; mid-chain branched alkyl sulfates as discussed in U.S. Pat. Nos. 6,020,303 and 6,060,443; and mid-chain branched alkyl alkoxy sulfates as discussed in U.S. Pat. Nos. 6,008,181 and 6,020,303.

Alkyl Sulfosuccinates—Sulfoacetate

Other suitable anionic surfactants are alkyl, preferably dialkyl, sulfosuccinates and/or sulfoacetate. The dialkyl sulfosuccinate may be a C₆₋₁₅ linear or branched dialkyl sulfosuccinate. The alkyl moieties may be symmetrical (i.e., the same alkyl moieties) or asymmetrical (i.e., different alkyl moieties). Preferably, the alkyl moiety is symmetrical.

Sulfonate Surfactants

The compositions of the invention will preferably comprise no more than 15% by weight, preferably no more than 10%, even more preferably no more than 5% by weight of the total composition, of a sulfonate surfactant. These include water-soluble salts or acids of C₁₀-C₁₄ alkyl or hydroxyalkyl, sulfonates; C₁₁-C₁₈ alkyl benzene sulfonates (LAS), modified alkylbenzene sulfonate (MLAS) as discussed in WO 99/05243, WO 99/05242, WO 99/05244, WO 99/05082, WO 99/05084, WO 99/05241, WO 99/07656, WO 00/23549 and WO 00/23548; methyl ester sulfonate (MES); and alpha-olefin sulfonate (AOS). These also include the paraffin sulfonates which may be monosulfonates and/or disulfonates, obtained by sulfonating paraffins of 10 to 20 carbon atoms. The sulfonate surfactants also include the alkyl glyceryl sulfonate surfactants.

Amphoteric and Zwitterionic Surfactants

The amphoteric and zwitterionic surfactant may be included in the compositions at a level of from 0.01% to 20%, preferably from 0.2% to 15%, more preferably 0.5% to 12% by weight. Suitable amphoteric and zwitterionic surfactants are amine oxides and betaines.

Most preferred are amine oxides, especially coco dimethyl amine oxide or coco amido propyl dimethyl amine oxide. Amine oxides may have a linear or mid-branched alkyl moiety. Typical linear amine oxides include water-soluble amine oxides of the formula R¹—N(R²)(R³)→O, wherein R¹ is a C₈₋₁₈ alkyl moiety; R² and R³ are independently selected from the group consisting of C₁₋₃ alkyl groups and C₁₋₃ hydroxyalkyl groups and preferably include methyl, ethyl, propyl, isopropyl, 2-hydroxethyl, 2-hydroxypropyl and 3-hydroxypropyl. The linear amine oxide surfactants may in particular include linear C₁₀-C₁₈ alkyl dimethyl amine oxides and linear C₈-C₁₂ alkoxy ethyl dihydroxy ethyl amine oxides. Preferred amine oxides include linear C₁₀, linear C₁₀-C₁₂, and linear C₁₂-C₁₄ alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the a carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2), such that the one alkyl moiety and the one alkyl branch are symmetric. As used herein “symmetric” means that n1-n2 is less than or equal to 5, preferably 4, most preferably from 0 to 4 carbon atoms in at least 50 wt %, more preferably at least 75 wt % to 100 wt % of the mid-branched amine oxides for use herein.

The amine oxide further comprises two moieties, independently selected from a C₁₋₃ alkyl, a C₁₋₃ hydroxyalkyl group, or a polyethylene oxide group containing an average of from about 1 to about 3 ethylene oxide groups. Preferably the two moieties are selected from a C₁₋₃ alkyl, more preferably both are selected as a C₁ alkyl.

Other suitable surfactants include betaines such alkyl betaines, alkylamidobetaine, amidazoliniumbetaine, sulfobetaine (INCI Sultaines) as well as phosphobetaines.

One preferred surfactant system is a mixture of anionic surfactant and amphoteric or zwiterionic surfactants in a ratio within the range of 1:1 to 5:1, preferably from 1:1 to 3.5:1.

For further information about amphoteric and zwitterionic surfactants, see WO 2012/015852.

Nonionic Surfactants

A nonionic surfactant, when present as a co-surfactant, may be in a typical amount of from 0.1% to 20%, preferably 0.5% to 15%, more preferably from 0.5% to 10% by weight of the liquid detergent composition. When present as the main surfactant, it may be in a typical amount of from 0.1% to 45%, preferably 15% to 40%, more preferably 20% to 35% by weight of the total composition. Suitable nonionic surfactants include the condensation products of aliphatic alcohols with from 1 to 25 moles of ethylene oxide. The alkyl chain of the aliphatic alcohol can either be straight or branched, primary or secondary, and generally contains from 8 to 22 carbon atoms. Particularly preferred are the condensation products of alcohols having an alkyl group containing from 10 to 18 carbon atoms, preferably from 10 to 15 carbon atoms with from 2 to 18 moles, preferably 2 to 15, more preferably 5 to 12 moles of ethylene oxide per mole of alcohol.

Also suitable are alkylpolyglycosides, alkylglycerol ethers and sorbitan esters, and fatty acid amide surfactants, e.g. as described in WO 2012/015852.

Cationic Surfactants

Cationic surfactants, when present in the composition, are present in an effective amount of e.g. 0.1% to 20% by weight of the liquid detergent composition. Suitable cationic surfactants are quaternary ammonium surfactants. Suitable quaternary ammonium surfactants are selected from the group consisting of mono C₆-C₁₆, preferably C₆-C₁₀ N-alkyl or alkenyl ammonium surfactants, wherein the remaining N positions are substituted by methyl, hydroxyethyl or hydroxypropyl groups. Another preferred cationic surfactant is a C₆-C₁₈ alkyl or alkenyl ester of a quaternary ammonium alcohol, such as quaternary chlorine esters.

Cationic Polymer

In a one embodiment, the liquid hand dishwashing compositions herein may comprise at least one cationic polymer for further enhanced skin benefits. The cationic polymer may be present an amount of from 0.001% to 10%, preferably from 0.01% to 5%, more preferably from 0.05% to 1%, by weight of the total composition. Suitable cationic polymers contain cationic nitrogen-containing moieties such as quaternary ammonium or cationic protonated amino moieties, and may have an average molecular weight of form about 5000 to about 10 million, preferably at least about 100,000, more preferably at least about 200,000, but preferably not more than about 3,000,000. See e.g. WO 2012/015852 for further information on the use of cationic polymers in liquid dishwashing compositions.

Humectant

The liquid dishwashing compositions may further comprise one or more humectants to provide additional hand skin mildness benefits. Alternatively, the compositions may be without a humectant.

When present, the humectant may be used in an amount of from 0.1% to 50%, preferably from 1% to 20%, more preferably from 1% to 10%, even more preferably from 1% to 6%, and most preferably from 2% to 5% by weight of the total composition.

Suitable humectants include those substances that exhibit an affinity for water and help enhance the absorption of water onto a substrate, preferably skin. Specific non-limiting examples of particularly suitable humectants include glycerol, diglycerol, polyethyleneglycol (PEG-4), propylene glycol, hexylene glycol, butylene glycol, (di)propylene glycol, glyceryl triacetate, polyalkyleneglycols, and mixtures thereof. Others can be polyethylene glycol ether of methyl glucose, pyrrolidone carboxylic acid (PCA) and its salts, pidolic acid and salts such as sodium pidolate, polyols like sorbitol, xylitol and maltitol, or polymeric polyols like polydextrose or natural extracts like quillaia, or lactic acid or urea. Also included are alkyl polyglycosides, polybetaine polysiloxanes, and mixtures thereof. Additional suitable humectants are polymeric humectants of the family of water soluble and/or swellable polysaccharides such as hyaluronic acid, chitosan and/or a fructose rich polysaccharide which is e.g. available as Fucogel®1000 (CAS-No. 178463-23-5) by SOLABIA S.

Humectants containing oxygen atoms are preferred over those containing nitrogen or sulphur atoms. More preferred humectants are polyols or are carboxyl-containing such as glycerol, diglycerol, sorbitol, propylene glycol, polyethylene glycol, butylene glycol; and/or pidolic acid and salts thereof, and most preferred are humectants selected from the group consisting of glycerol, sorbitol, sodium lactate and urea, or mixtures thereof.

Cleaning Polymer

The liquid hand dishwashing compositions herein may optionally further comprise an alkoxylated polyethyleneimine polymer. The composition may comprise from 0.01% to 10%, preferably from 0.01% to 2%, more preferably from 0.1% to 1.5%, even more preferably from 0.2% to 1.5% by weight of the total composition of an alkoxylated polyethyleneimine polymer e.g. as described in WO 2007/135645.

The alkoxylated polyethyleneimine polymer may have a polyethyleneimine backbone having from 400 to 10000 weight average molecular weight, preferably from 400 to 7000 weight average molecular weight, alternatively from 3000 to 7000 weight average molecular weight.

The alkoxylation of the polyethyleneimine backbone includes: (1) one or two alkoxylation modifications per nitrogen atom, depending on whether the modification occurs at an internal nitrogen atom or at an terminal nitrogen atom, in the polyethyleneimine backbone, the alkoxylation modification consisting of the replacement of a hydrogen atom by a polyalkoxylene chain having an average of about 1 to about 40 alkoxy moieties per modification, wherein the terminal alkoxy moiety of the alkoxylation modification is capped with hydrogen, a C₁-C₄ alkyl or mixtures thereof; (2) a substitution of one C₁-C₄ alkyl moiety or benzyl moiety and one or two alkoxylation modifications per nitrogen atom, depending on whether the substitution occurs at an internal nitrogen atom or at a terminal nitrogen atom in the polyethyleneimine backbone, the alkoxylation modification consisting of the replacement of a hydrogen atom by a polyalkoxylene chain having an average of about 1 to about 40 alkoxy moieties per modification wherein the terminal alkoxy moiety is capped with hydrogen, a C₁-C₄ alkyl or mixtures thereof; or (3) a combination thereof.

The composition may further comprise amphiphilic graft polymers based on water soluble polyalkylene oxides (A) as a graft base and side chains formed by polymerization of a vinyl ester component (B), said polymers having an average of not more than 1 graft site per 50 alkylene oxide units and mean molar mass Mw of from 3,000 to 100,000 as described in WO 2007/138053.

Magnesium Ions

The optional presence of magnesium ions may be utilized in the detergent composition when the compositions are used in softened water that contains few divalent ions. When utilized, the magnesium ions preferably are added as a hydroxide, chloride, acetate, sulfate, formate, oxide or nitrate salt to the compositions of the invention. When included, the magnesium ions are present at an active level of from 0.01% to 1.5%, preferably from 0.015% to 1%, more preferably from 0.025% to 0.5%, by weight of the liquid detergent composition.

Solvent

The present compositions may optionally comprise a solvent. Suitable solvents include C₄₋₁₄ ethers and diethers, glycols, alkoxylated glycols, C₆-C₁₆ glycol ethers, alkoxylated aromatic alcohols, aromatic alcohols, aliphatic branched alcohols, alkoxylated aliphatic branched alcohols, alkoxylated linear C₁-C₅ alcohols, linear C₁-C₅ alcohols, amines, C₈-C₁₄ alkyl and cycloalkyl hydrocarbons and halohydrocarbons, and mixtures thereof. When present, the liquid detergent composition will contain from 0.01% to 20%, preferably from 0.5% to 20%, more preferably from 1% to 10% by weight of the liquid detergent composition of a solvent. These solvents may be used in conjunction with an aqueous liquid carrier, such as water, or they may be used without any aqueous liquid carrier being present.

Hydrotrope

The liquid detergent compositions of the invention may optionally comprise a hydrotrope in an effective amount so that the liquid detergent compositions are appropriately compatible in water. Suitable hydrotropes for use herein include anionic-type hydrotropes, particularly sodium, potassium and ammonium xylene sulfonate, sodium, potassium and ammonium toluene sulfonate, sodium, potassium and ammonium cumene sulfonate, and mixtures thereof, and related compounds. The liquid detergent compositions of the invention may comprise from 0% to 15% by weight of the total liquid detergent composition of a hydrotrope, or mixtures thereof, preferably from 1% to 10%, most preferably from 3% to 10% by weight of the total liquid hand dishwashing composition.

Polymeric Suds Stabilizer

The compositions of the invention may optionally contain a polymeric suds stabilizer. These polymeric suds stabilizers provide extended suds volume and suds duration of the liquid detergent compositions. These polymeric suds stabilizers may be selected from homopolymers of (N,N-dialkylamino) alkyl esters and (N,N-dialkylamino) alkyl acrylate esters.

The weight average molecular weight of the polymeric suds boosters, determined via conventional gel permeation chromatography, is from 1,000 to 2,000,000, preferably from 5,000 to 1,000,000, more preferably from 10,000 to 750,000, more preferably from 20,000 to 500,000, even more preferably from 35,000 to 200,000. The polymeric suds stabilizer can optionally be present in the form of a salt, either an inorganic or organic salt.

One preferred polymeric suds stabilizer is an (N,N-dimethylamino) alkyl acrylate ester. Other preferred suds boosting polymers are copolymers of hydroxypropylacrylate/dimethyl aminoethylmethacrylate (copolymer of HPA/DMAM).

When present in the compositions, the polymeric suds booster/stabilizer may be present from 0.01% to 15%, preferably from 0.05% to 10%, more preferably from 0.1% to 5%, by weight of the liquid detergent composition.

Another preferred class of polymeric suds booster polymers is hydrophobically modified cellulosic polymers having a number average molecular weight (Mw) below 45,000; preferably between 10,000 and 40,000; more preferably between 13,000 and 25,000. The hydrophobically modified cellulosic polymers include water soluble cellulose ether derivatives, such as nonionic and cationic cellulose derivatives. Preferred cellulose derivatives include methylcellulose, hydroxypropyl methylcellulose, hydroxyethyl methylcellulose, and mixtures thereof.

Diamines

Another optional ingredient of the compositions of the invention is a diamine. Since liquid detergent compositions show considerable variation, the composition may contain 0% to 15%, preferably 0.1% to 15%, preferably 0.2% to 10%, more preferably 0.25% to 6%, more preferably 0.5% to 1.5% by weight of said composition of at least one diamine.

Preferred organic diamines are those in which pK1 and pK2 are in the range of 8.0 to 11.5, preferably in the range of 8.4 to 11, even more preferably from 8.6 to 10.75. Preferred materials include 1,3-bis(methylamine)-cyclohexane (pKa=10 to 10.5), 1,3 propane diamine (pK1=10.5; pK2=8.8), 1,6 hexane diamine (pK1=11; pK2=10), 1,3 pentane diamine (DYTEK AO) (pK1=10.5; pK2=8.9), 2-methyl 1,5 pentane diamine (DYTEK A®) (pK1=11.2; pK2=10.0). Other preferred materials include primary/primary diamines with alkylene spacers ranging from C₄ to C₅.

Carboxylic Acid

The liquid detergent compositions of the invention may comprise a linear or cyclic carboxylic acid or salt thereof to improve the rinse feel of the composition. The presence of anionic surfactants, especially when present in higher amounts in the region of 15-35% by weight of the composition, results in the composition imparting a slippery feel to the hands of the user and the dishware. This feeling of slipperiness is reduced when using the carboxylic acids as defined herein.

Carboxylic acids useful herein include C₁₋₆ linear or at least 3 carbon containing cyclic acids. The linear or cyclic carbon-containing chain of the carboxylic acid or salt thereof may be substituted with a substituent group selected from the group consisting of hydroxyl, ester, ether, aliphatic groups having from 1 to 6, more preferably 1 to 4 carbon atoms, and mixtures thereof.

Preferred carboxylic acids are those selected from the group consisting of salicylic acid, maleic acid, acetyl salicylic acid, 3-methyl salicylic acid, 4-hydroxy isophthalic acid, dihydroxyfumaric acid, 1,2,4-benzene tricarboxylic acid, pentanoic acid and salts thereof and mixtures thereof. Where the carboxylic acid is in the salt form, the cation of the salt is preferably selected from alkali metal, alkaline earth metal, monoethanolamine, diethanolamine or triethanolamine and mixtures thereof.

The carboxylic acid or salt thereof, when present, is preferably present at the level of from 0.1% to 5%, more preferably from 0.2% to 1% and most preferably from 0.25% to 0.5%, by weight of the total composition.

Other Components

The liquid detergent compositions herein can further comprise various other optional ingredients suitable for use in liquid detergent compositions such as perfume, dyes, opacifiers, other enzymes, chelants, pH buffering means and rheology modifiers, including those of the polyacrylate, polysaccharide or polysaccharide derivative type and/or a combination of a solvent and a polycarboxylate polymer.

Thickness

The liquid hand dishwashing compositions herein are typically thickened and preferably have a viscosity from 50 to 5000 centipoises (50-5000 mPa*s), more preferably from 100 to 4000 centipoises (100-4000 mPa*s), even more preferably from 500-3500 centipoises (500-3500 mPa*s), and most preferably from 800 to 3000 centipoises (800-3000 mPa*s) at 20^{s-1} and 20° C. Viscosity can be determined by conventional methods known in the art, for example measured using an AR 550 rheometer from TA Instruments using a plate steel spindle at 40 mm diameter and a gap size of 500 μm. The high shear viscosity at 20^{s-1} and low shear viscosity at 0.05^{s-1} can be obtained from a logarithmic shear rate sweep from 0.1^{s-1} to 25^{s-1} in 3 minutes time at 20° C. The preferred rheology may be achieved using internal existing structuring with detergent ingredients or by employing an external rheology modifier and/or a crystalline structurant, which provides the composition with a pseudoplastic or shear thinning rheology profile and with time-dependent recovery of viscosity after shearing (thixotropy).

Crystalline Structurants

The compositions of the invention may further comprise one or more crystalline structurants, which are materials that form a thread-like structuring system and/or an insoluble particle network throughout the matrix of the composition. The crystalline structurants may be crystallized in situ within the aqueous liquid matrix of the composition or within a pre-mix which is used to form such an aqueous liquid matrix. It has been found that the network generated by the crystalline wax structurant prevents the hydrophobic emollient droplets from coalescing and phase splitting in the product, thereby providing excellent stability of a hand dishwashing liquid composition.

When present, said crystalline structurant will typically be comprised at a level of from 0.02% to 5%, preferably from 0.025% to 3%, more preferably from 0.05% to 2%, most preferably from 0.1% to 1.5% by weight of the total composition. Preferred crystalline structurants are: hydroxyl-containing crystalline structuring agents such as a hydroxyl-containing fatty acid, fatty ester or fatty soap wax-like materials or the like such as the ones described in U.S. Pat. No. 6,080,707.

Other suitable crystalline structurants include C₁₀₋₂₂ ethylene glycol fatty acid esters. C₁₀₋₂₂ ethylene glycol fatty acid esters can be used alone or in combination with another crystalline structurant such as hydrogenated castor oil. Typical examples are monoesters and/or diesters of ethylene glycol, propylene glycol, diethylene glycol, dipropylene glycol, triethylene glycol or tetraethylene glycol with fatty acids containing from about 6 to about 22, preferably from about 12 to about 18 carbon atoms, such as caproic acid, caprylic acid, 2-ethyhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, erucic acid, and mixtures thereof.

For further information on suitable crystalline structurants, see WO 2012/015852.

Additional Enzymes

The detergent composition may comprise one or more additional enzymes such as an amylase, an arabinase, a carbohydrase, a cellulase (e.g., endoglucanase), a cutinase, a deoxyribonuclease, a galactanase, a haloperoxygenase, a lipase, a mannanase, an oxidase, e.g., a laccase and/or peroxidase, a pectinase, a pectin lyase, an additional protease, a xylanase, a xanthanase, a xyloglucanase or an oxidoreductase.

When the composition comprises one or more additional enzymes, the additional enzyme is preferably an amylase and/or a lipase, in particular an amylase.

The properties of the selected enzyme(s) should be compatible with the selected detergent (e.g. pH-optimum, compatibility with other enzymatic and non-enzymatic ingredients, etc.).

Proteases

The composition may, in addition to the protease as disclosed herein, comprise one or more additional proteases including those of bacterial, fungal, plant, viral or animal origin. Proteases of microbial origin are preferred. The protease may be an alkaline protease, such as a serine protease or a metalloprotease. A serine protease may for example be of the S1 family, such as trypsin, or the S8 family such as subtilisin. A metalloprotease may for example be a thermolysin from, e.g., family M4 or another metalloprotease such as those from M5, M7 or M8 families.

Examples of metalloproteases are the neutral metalloproteases as described in WO 2007/044993 (Genencor Int.) such as those derived from Bacillus amyloliquefaciens.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase®, Esperase®, Progress® Uno and Progress® Excel (Novozymes A/S), those sold under the tradenames Maxatase®, Maxacal®, Maxapem®, Purafect®™, Purafect® Ox, Purafect® OxP, Purafect Prime®, Puramax®, FN2®, FN3®, FN4®, Excellase®, Excellenz P1000™, Excellenz P1250™, Eraser®, Preferenz® P100, Preferenz® P110, Effectenz P1000™, Effectenz P1050™, Effectenz P2000™, Purafast®, Properase®, Opticlean® and Optimase® (Danisco/DuPont), Axapem™(Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase, in particular the S54V variant used in the commercial product Gentle Power Bleach from Huntsman Textile Effects Pte Ltd (WO 2010/100028).

Amylases

Suitable amylases which can be used together with the protease may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/10355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylases comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M 197T;

H156Y+A181T+N190F+A209V+Q264S; or

G48A+T49I+G107A+H156Y+A181T+N190F+I201F+A209V+Q264S.

Other suitable amylases are amylases having the sequence of SEQ ID NO: 6 in WO 99/19467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/23873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K,

wherein the variants are C-terminally truncated and optionally further comprise a substitution at position 243 and/or a deletion at position 180 and/or position 181.

Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or a deletion in position R178 and/or S179 or of T180 and/or G181. Most preferred amylase variants of SEQ ID NO: 1 comprise the substitutions:

E187P+I203Y+G476K

E187P+I203Y+R458N+T459S+D460T+G476K

and optionally further comprise a substitution at position 241 and/or a deletion at position 178 and/or position 179.

Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478.

More preferred variants of SEQ ID NO: 1 are those having the substitution in one of more of the following positions: N21D, D97N, V128 K177L, M200L, L204YF, E242QA, G477K and G478K and/or a deletion in position R179 and/or S180 or of I181 and/or G182. Most preferred amylase variants of SEQ ID NO: 1 comprise the substitutions N21D+D97N+V128I, and optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087. Commercially available amylases include Duramyl™, Termamyl™, Fungamyl™, Stainzyme™, Stainzyme Plus™, Natalase™, Liquozyme X, BAN™, Amplify® and Amplify® Prime (from Novozymes A/S), and Rapidase™, Purastar™/Effectenz™, Powerase, Preferenz S1000, Preferenz S100 and Preferenz S110 (from Genencor International Inc./DuPont).

One preferred amylase is a variant of the amylase having SEQ ID NO: 13 in WO 2016/180748 with the alterations H1*+N54S+ V56T+ K72R+G109A+ F113Q+ R116Q+ W167F+ Q172G+ A174S+ G182*+D183*+ G184T+ N195F+ V206L+ K391A+ P473R+ G476K.

Another preferred amylase is a variant of the amylase having SEQ ID NO: 1 in WO 2013/001078 with the alterations D183*+G184*+W140Y+N195F+V206Y+Y243F+E260G+G304R+G476K.

Another preferred amylase is a variant of the amylase having SEQ ID NO: 1 in WO 2018/141707 with the alterations H1*+G7A+G109A+W140Y+G182*+D183*+N195F+V206Y+Y243F+E260G+N280S+G304R+E391A+G476K.

A further preferred amylase is a variant of the amylase having SEQ ID NO: 1 in WO 2017/191160 with the alterations L202M+T246V.

### Deoxyribonucleases (DNases)

Suitable deoxyribonucleases (DNases) are any enzyme that catalyzes the hydrolytic cleavage of phosphodiester linkages in the DNA backbone, thus degrading DNA. A DNase which is obtainable from a bacterium is preferred, in particular a DNase which is obtainable from a species of Bacillus is preferred; in particular a DNase which is obtainable from Bacillus subtilis or Bacillus licheniformis is preferred. Examples of such DNases are described in WO 2011/098579 and WO 2014/087011.

### Oxidoreductases

In one embodiment, the composition may comprise an oxidoreductase, which are enzymes that catalyze reduction-oxidation reactions. A preferred oxidoreductase is a superoxide dismutase.

Uses and Cleaning Methods

The present invention is also directed to use of the liquid detergent compositions for cleaning, in particular for hard surface cleaning such as dishwashing, in particular for hand dishwashing.

This aspect further relates to a method of cleaning, especially for cleaning hard surfaces or for dishwashing, comprising contacting the hard surface or dishes with the liquid detergent composition under conditions suitable for cleaning the surface or dishes. In a particular embodiment, the method of cleaning is for hand dishwashing and comprises contacting dishes with a detergent composition comprising a protease as described herein under conditions suitable for cleaning the dishes.

In one embodiment, the invention relates to use a protease for preparing a liquid detergent composition suitable for hand dishwashing, wherein the composition provides hand skin care benefits, wherein the protease is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2. In this embodiment, the protease may be any of those described in more detail above.

In one embodiment, the invention relates to use a protease as disclosed herein for preparing a liquid detergent composition suitable for hand dishwashing, wherein the composition provides hand skin care benefits, and wherein use of the composition in hand dishwashing provides at least one hand skin care benefit selected from corneocyte removal, reduced transepidermal water loss, an improved hand skin barrier, and improved hand skin water retention.

It will be understood that the protease for these uses and cleaning methods may be any of the proteases described in more detail elsewhere herein.

The present invention is further described by the following examples that should not be construed as limiting the scope of the invention.

### EXAMPLES

### Example 1

### Skin Care Benefits of a Composition of the Invention

Studies were performed to assess the skin care benefits of a composition of the invention comprising a protease.

The studies were: 1) a randomized, single-blind, one single use, intra-group and inter-group controlled, evaluation of the skin care benefit of the test product on skin texture (dead corneocyte removal and skin smoothness improvement); and 2) a randomized, single-blind, one single use, inter-group controlled evaluation of the moisture retention benefit on hand skin gained by using the test product.

The studies compared a commercial hand dishwashing detergent (Fairy hand dishwashing detergent) either alone (no enzymes) or with 0.2% by weight of a protease, where the protease is a variant of SEQ ID NO: 1 with the substitutions S9E, N43R, N76D, V205I, Q206L, Y209W, S259D, N261W and L262E, where position numbers are based on the numbering of SEQ ID NO: 2. The protease was obtained from Novozymes A/S, Denmark, and was added to the commercial detergent in the form of a liquid.

Test Procedure

Thirty subjects were recruited and completed the study in Shanghai, China. All subjects were provided with a thorough explanation of the study and gave oral and written informed consent in accordance with local laws and regulations. Participation was voluntary, and subjects were free to leave the study at any time. All 30 participants completed the study.

The recruited subjects were not allowed to have used any cosmetics, topical medications, or other hand care products at least three days before the test. Subjects were not allowed to use hand cream products on the morning of the test or to wash hands one hour before the test.

For determination of a baseline, subjects were asked to sit in a prescribed environment for 30 minutes without any beverages allowed. The forearms were then exposed and placed under test conditions, kept relaxed. Two test regions were selected and marked on the back of each hand. The textures of the hand back were evaluated using a Visioscan® VC98 instrument (Courage & Khazaka, Germany), and the water loss rate was measured on hand skin with a Tewameter® instrument (Courage & Khazaka, Germany).

Product application: Two researchers helped each subject soak and wash their hands with the product at the same time. The two hands were treated with different products (with and without protease). Hands of subjects were soaked in product solution for 5 minutes, after which the staff applied 10 g of product foam (extruded from a foaming bottle) with a sponge to wash the hands of subjects for 15 minutes (20 times/minute). Subsequently, the subjects washed their hands in clear water (temperature 30-35° C.), and gently removed the excess water from their hands with another dry sponge. The water loss value was then measured with the Tewameter® instrument for 180 seconds to calculate the water retention time immediately after drying.

The subjects' hands were kept dry at room temperature for 60 min, after which evaluation of hand surface texture and smoothness assessments with the VC98® instrument and a hydration test on water loss rate with the Tewameter® instrument were performed. After the instrument evaluation, subjects were asked to fill out a self-assessment questionnaire.

The following parameters were measured:

(1) Skin texture: The VC98® instrument was used to test two regions on the back of the hand, before and after product use (60 minutes later). The mean value of the two areas was calculated for each hand. The corneocyte parameter (corneocytes area ratio) was analyzed using Image-Pro Plus software (Media Cybernetics, Inc.).

(2) Transepidermal water loss: A VapoMeter (Delfin, Finland) was used to test two regions on the back of the hand before and after use (60 minutes later). The mean value of two areas was calculated for each hand.

(3) Skin water retention time: The Tewameter0 instrument, used for 180 seconds, determined the water retention time. An average of the two areas on each hand was calculated.

For each parameter, a delta value was determined for the test product hand (i.e. detergent+protease) and the control hand (i.e. detergent alone).

Results

Corneocyte Area Ratio:

The delta value of the corneocyte area (no units) was −0.0757 for the detergent with 0.2% protease as compared to −0.0277 for the detergent alone (P=0.002).

A larger corneocyte area ratio means that there are more corneocytes on the skin. Thus, a larger delta value of corneocyte area ratio indicates better corneocyte removal performance. Addition of the protease has therefore been shown to have a positive effect on corneocyte removal.

Transepidermal Water Loss (TEWL):

The delta value of the TEWL (g/m²h) was −7.5 for the detergent with 0.2% protease as compared to 3.8 for the detergent alone (P<0.001).

The delta value of TEWL of 3.8 g/m²h for the control hand (detergent alone) represents a loss of water, as the skin barrier functions poorly after washing with the detergent. In contrast, the delta value of TEWL of −7.5 g/m²h for the test product hand (detergent+protease) represents a gain of water, showing that in spite of the harmful effect of the detergent to the hand skin barrier, this could be converted to a significant barrier protection benefit by addition of protease.

Skin Water Retention Time:

The delta value of the skin water retention time (sec) was 120.2 for the detergent with 0.2% protease as compared to 83.6 for the detergent alone (P=0.021).

A longer skin water retention time indicates a better moisture preservation effect on hand skin. Compared with the hand washed with detergent only, the skin water retention time is longer on the other hand washed with detergent+protease, indicating that the addition of the 0.2% protease has a positive effect on moisture preservation on hand skin.

Questionnaire Results:

For each of the following questions, more participants selected “product area” (hand washed with detergent+protease) compared to “control area” (detergent alone):
Which hand is smoother after washing with the product than before?
Which hand is more moist and hydrated after washing than before?
Which hand is softer after washing with the product than before?
Which hand has better skin color after washing with the product than before?
Which hand has less dead or flaky skin after washing with the product than before?
Which product makes your skin feel softer and less irritating than before?
Which product gives you a more natural exfoliating effect than before?

For the following question, more participants selected “control area” (detergent alone) compared to “product area” (hand washed with detergent+protease):
Which product makes your hand skin feel tight after use than before?

Conclusions

Under the study conditions with 30 subjects, it was observed:

Compared with the hand washed with detergent only, the corneocytes on the other hand washed with detergent+protease were significantly reduced (P=0.002), indicating that addition of 0.2% of the protease has a positive effect on corneocyte removal.

Compared with the hand washed with detergent only, transepidermal water loss is significantly reduced (P<0.001) on the other hand washed with detergent+protease, indicating that the addition of 0.2% of the protease has a positive effect on the hand skin barrier.

Compared with the hand washed with detergent only, the skin water retention time was significantly longer (P=0.021) for the other hand washed with detergent+protease, indicating that the addition of 0.2% of the protease can improve water retention time on hand skin.

Further, based on the answers to the questionnaire, it was found that more subjects experienced improved moisture and hydration after wash using the detergent with 0.2% protease compared with the hand washed by detergent only. It was also found that more subjects said that the skin felt less tight on the hand washed by detergent with 0.2% protease compared with the other hand washed by detergent only.

### Example 2

### Corneocyte Removal by a Composition of the Invention

A study was performed to assess corneocyte removal of a hand dishwashing composition of the invention.

Test Procedure

A solution of a commercial hand dishwashing detergent (Blue Moon green tea hand dish wash detergent; 10 g dishwashing detergent per liter of tap water) was prepared in advance. Five subjects with visibly flaky hands were recruited for the test. One hand was treated with the detergent only, and the other hand was treated with the detergent containing one of five different proteases. The proteases used were the following (for proteases 1-4, the position numbers are based on the numbering of SEQ ID NO: 2):
1. SEQ ID NO: 1 with the substitutions S9E+N43R+N76D+V205I+Q206L+Y209W+S259D+N261W+L262E
2. SEQ ID NO: 1
3. SEQ ID NO: 1 with the substitutions S9E+N43R+N76D+N185E+5188E+Q191N+A194P+Q206L+Y209W+S259D+L262E
4. SEQ ID NO: 1 with the alteration S99AD (i.e. the substitution S99A and insertion of D)
5. SEQ ID NO: 5

The protease concentration in the dishwashing liquid was 0.3% by weight.

Corneocytes were harvested from the left and right hands using transparent Scotch tape. It was found that corneocytes were well distributed on both hands. Four replicate tapes were used on each hand for the test.

Tapes with corneocytes were incubated with 500 ml detergent only or detergent+0.3% protease solutions at 40° C. for 10 min. Residual corneocytes on the tapes were stained with 1 mg/mL Toluidine Blue O for 15 min at room temperature, then rinsed with tap water after coloring.

Residual corneocytes were measured at R460 nm on a DigiEye system. The benefit of addition of protease, expressed as enhancement of corneocyte removal, was calculated as:

Corneocyte removal benefit (%)=[residual corneocytes (with protease)−residual corneocytes (detergent only)]/corneocytes before wash

TABLE 1
Corneocyte removal by 0.3% protease in hand dishwashing detergent

| Protease | | Corneocyte removal benefit (%) |
|---|---|---|
| 1 | SEQ ID NO: 1 + S9E + N43R + N76D + V205I + Q206L + | 50.0 |
| | Y209W + S259D + N261W + L262E | |
| 2 | SEQ ID NO: 1 | 25.5 |
| 3 | SEQ ID NO: 1 + S59E + N43R + N76D + N185E + S188E + | 32.1 |
| | Q191N + A194P + Q206L + Y209W + S259D + L262E | |
| 4 | SEQ ID NO: 1 + S99AD | 32.1 |
| 5 | SEQ ID NO: 5 | 34.7 |

It may be seen from the table above that all of the tested proteases provide a corneocyte removal benefit, with protease 2 (Savinase®) providing less of a benefit than the others, and with protease 1 providing a substantially better corneocyte removal than the other tested proteases.

1. A liquid detergent composition suitable for hand dishwashing, comprising at least one protease, wherein the protease is a variant of the polypeptide of SEQ ID NO: 1 comprising two or more substitutions selected from the group consisting of S9E, N43R, N76D, Q206L, Y209W, S259D and L262E, wherein the variant has protease activity and has at least 80%, at least 85%, at least 90% or at least 95% but less than 100% sequence identity to SEQ ID NO: 1, and wherein position numbers are based on the numbering of SEQ ID NO: 2.

2. The composition of claim 1, wherein the protease is a variant of the polypeptide of SEQ ID NO: 1 comprising 3, 4, 5, 6 or all of the substitutions selected from the group consisting of S9E, N43R, N76D, Q206L, Y209W, S259D and L262E, wherein position numbers are based on the numbering of SEQ ID NO: 2.

3. The composition of claim 1, wherein the protease is a variant of the polypeptide of SEQ ID NO: 1 comprising 3, 4, 5, 6, 7, 8 or all of the substitutions selected from the group consisting of S9E, N43R, N76D, V205I, Q206L, Y209W, S259D, N261W and L262E, wherein position numbers are based on the numbering of SEQ ID NO: 2.

4. The composition of claim 3, wherein the protease variant comprises or consists of SEQ ID NO: 1 with the substitutions S9E, N43R, N76D, V205I, Q206L, Y209W, S259D, N261W and L262E, wherein position numbers are based on the numbering of SEQ ID NO: 2.

5. A liquid detergent composition suitable for hand dishwashing, comprising at least one protease, wherein the protease is selected from the group consisting of:
(a) the polypeptide of SEQ ID NO: 1;
(b) a variant of the polypeptide of SEQ ID NO: 1 with the mutation S99SE, wherein position numbers are based on the numbering of SEQ ID NO: 2;
(c) a variant of the polypeptide of SEQ ID NO: 1 with the mutation S99AD, wherein position numbers are based on the numbering of SEQ ID NO: 2;
(d) a variant of the polypeptide of SEQ ID NO: 1 with the substitutions Y167A+R170S+A194P, wherein position numbers are based on the numbering of SEQ ID NO: 2;
(e) a variant of the polypeptide of SEQ ID NO: 1 with the substitutions S99D+S101R/E+S103A+V104I+G160S; for example a variant of SEQ ID NO: 1 with the substitutions S3T+V4I+S99D+S101E+S103A+V104I+G160S+V205I, wherein position numbers are based on the numbering of SEQ ID NO: 2;
(f) a variant of the polypeptide of SEQ ID NO: 1 comprising 3, 4, 5, 6, 7, 8, 9, 10 or all of the substitutions selected from the group consisting of S9E, N43R, N76D, N185E, S188E, Q191N, A194P, Q206L, Y209W, S259D and L262E, for example a variant comprising or consisting of SEQ ID NO: 1 with the substitutions S9E, N43R, N76D, N185E, S188E, Q191N, A194P, Q206L, Y209W, S259D and L262E, wherein position numbers are based on the numbering of SEQ ID NO: 2;
(g) the polypeptide of SEQ ID NO: 3 or a variant thereof, for example a variant of SEQ ID NO: 3 with the substitutions S27K+N109K+S111E+S171E+S173P+G174K+S175P+F180Y+G182A+L184F+Q198E+N199K+T297P, wherein position numbers are based on the numbering of SEQ ID NO: 3; and
(h) the polypeptide of SEQ ID NO: 4.

6. The composition of claim 5, comprising the protease in an amount of from about 0.001% w/w to about 4% w/w, such as about 0.01% w/w to about 2% w/w, such as about 0.05% w/w to about 1% w/w, such as about 0.1% w/w to about 0.5% w/w, wherein percentages are enzyme protein by weight of the composition.

7. The composition of claim 5, wherein use of the composition in hand dishwashing provides at least one hand skin care benefit selected from corneocyte removal, reduced transepidermal water loss, an improved hand skin barrier, and improved hand skin water retention.

8. A liquid hand dishwashing detergent composition comprising a protease of claim 5, wherein the composition provides at least one hand skin care benefit selected from corneocyte removal, reduced transepidermal water loss, an improved hand skin barrier, and improved hand skin water retention.

9. The composition of claim 8, wherein the protease comprises or consists of SEQ ID NO: 1 with the substitutions S9E, N43R, N76D, V205I, Q206L, Y209W, S259D, N261W and L262E, wherein position numbers are based on the numbering of SEQ ID NO: 2.

10. (canceled)

11. (canceled)

12. A method for preparing a hand dishwashing detergent composition of claim 5 with skin care benefits, or for providing skin care benefits to a hand dishwashing detergent composition, the method comprising adding to the composition the at least one protease.

13. The method of claim 12, wherein the protease comprises or consists of SEQ ID NO: 1 with the substitutions S9E, N43R, N76D, V205I, Q206L, Y209W, S259D, N261W and L262E, wherein position numbers are based on the numbering of SEQ ID NO: 2.

14. (canceled)

15. A method of cleaning, e.g. for hand dishwashing or hard surface cleaning, comprising contacting dishes or a hard surface to be cleaned with a detergent composition of claim 5 under conditions suitable for cleaning the dishes or surface.

16. The composition of claim 1, comprising the protease in an amount of from about 0.001% w/w to about 4% w/w, such as about 0.01% w/w to about 2% w/w, such as about 0.05% w/w to about 1% w/w, such as about 0.1% w/w to about 0.5% w/w, wherein percentages are enzyme protein by weight of the composition.

17. The composition of claim 1, wherein use of the composition in hand dishwashing provides at least one hand skin care benefit selected from corneocyte removal, reduced transepidermal water loss, an improved hand skin barrier, and improved hand skin water retention.

18. A liquid hand dishwashing detergent composition comprising a protease of claim 1, wherein the composition provides at least one hand skin care benefit selected from corneocyte removal, reduced transepidermal water loss, an improved hand skin barrier, and improved hand skin water retention.

19. A method for preparing a hand dishwashing detergent composition of claim 1 with skin care benefits, or for providing skin care benefits to a hand dishwashing detergent composition, the method comprising adding to the composition the at least one protease.

20. A method of cleaning, e.g. for hand dishwashing or hard surface cleaning, comprising contacting dishes or a hard surface to be cleaned with a detergent composition of claim 1 under conditions suitable for cleaning the dishes or surface.


SEQUENCE LISTING (5 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 311 aa, Bacillus sp. [sequence omitted]

SEQ ID NO: 4 - protein, 274 aa, Artificial Sequence
  Bacillus licheniformis variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 5 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ